(19) United States
(12) Patent Application Publication
(10) Pub. No.: US 2025/0235196 A1
(43) Pub. Date: Jul. 24, 2025

(54) Bowel Gripping And Distraction Device

(71) Applicant: The Regents of the University of Michigan, Ann Arbor, MI (US)

(72) Inventors: Thomas MARTEN, Canton, MI (US); gardner Yost, Ann Arbor, MI (US); Diann Brei, millford, MI (US); Matthew Ralls, Dexter, MI (US); Jonathan E. Luntz, Ann Arbor, MI (US); Jeffrey Stephen Plott, Algonac, MI (US)

(21) Appl. No.: 19/027,933

(22) Filed: Jan. 17, 2025

Related U.S. Application Data
(60) Provisional application No. 63/624,441, filed on Jan. 24, 2024.

(51) Int. Cl. A61B 17/02

(52) U.S. Cl. CPC A61B 17/0281

(57) ABSTRACT

A bowel gripping and distraction device includes a first bowel gripping feature configured to be inflatable to engage a bowel, the first gripping feature including a plurality of radially extending fins. A second bowel gripping feature is configured to be inflatable to engage the bowel, the first gripping feature including a plurality of radially extending fins. An elongation system is operable to extend a relative distance between the first bowel gripping feature and the second bowel gripping feature to apply a tensile force to the bowel of a patient, the tensile force being configured to encourage growth of the bowel through mechanotransductive treatment.

### CROSS-REFERENCE TO RELATED APPLICATIONS

This application claims the benefit of U.S. Provisional Application No. 63/624,441, filed on Jan. 24, 2024

### FIELD

The present disclosure relates to bowel extension devices and, more particularly, relates to a device that uses mechanical pressure to encourage gastrointestinal expansion and growth to treat short bowel syndrome.

### BACKGROUND

This section provides background information related to the present disclosure which is not necessarily prior art.

Short bowel syndrome (SBS) is a condition in which congenital, infectious, or traumatic injuries result in loss of bowel length and this decreased intestinal surface area is unable to absorb sufficient nutrients to sustain life. Parenteral nutrition (PN), in which calories and nutrients are delivered intravenously, is life sustaining but associated with many complications including infection, liver disease, and even death. Current treatments for SBS are inadequate with cure rates reaching only 30-40%.

The principles of the present teachings provide a mechanotransductive bowel extender device that can be used to correct short bowel syndrome by applying tension to the bowel tissue which, via the process of mechanotransduction, grows in length in response to the applied tension. Mechanotransduction in cellular biology is a mechanism by which cells convert mechanical stimulus into electrochemical activity. In the case of the growth of the bowel, such a device is placed within the small bowel and applies tension over a period of several days or weeks by attaching to the lumen of the bowel at two different locations and moving those two points apart, thereby stretching the tissue to stimulate growth of the bowel.

### SUMMARY

This section provides a general summary of the disclosure and is not a comprehensive disclosure of its full scope or all of its features.

According to the principles of the present teachings, a bowel engagement element is provided having a gripping material and a balloon. In some embodiments, the gripping material is preferably made of a biocompatible urethane, TPU, or other flexible material. In some embodiments, the wall thickness of the base of the gripping material is preferably such that it is thin enough where the pressure of balloon inflation causes material elongation of the wall of the gripping material. The gripping material can include a plurality of angled fins. In some embodiments, when the balloon is in a deflated state, the angled fins remain flexible, allowing them to easily deflect and allow the device to translate within the bowel, even if the fins are pointing against the direction of travel within the bowel. In some embodiments, when the balloon is inflated, tension is created on the fin base. This tension reduces the fin's ability to deflect, allowing it to more effectively grip the bowel. Further, in some embodiments, as the balloon inflates, it changes the angle of attack for the fin on the gripping material, further improving its ability attach to the bowel. Also, as the balloon inflates, it increases the diameter of the gripping material, causing it to press more firmly against the bowel (or tissue). The gripping material can be created in multiple configurations. For example, there can be strips of gripping material, as shown. In some embodiments, different numbers of strips can be used. In some embodiments, these strips could be rectangular or other shapes. The number of strips can vary. The gripping material could be separate from the balloon or integral to the balloon.

According to an aspect of the present disclosure, a bowel gripping and distraction device includes a first bowel gripping feature configured to be inflatable to engage a bowel, the first gripping feature including a plurality of radially extending fins. A second bowel gripping feature is configured to be inflatable to engage the bowel, the first gripping feature including a plurality of radially extending fins. An elongation system is operable to extend a relative distance between the first bowel gripping feature and the second bowel gripping feature to apply a tensile force to the bowel of a patient, the tensile force being configured to encourage growth of the bowel through mechanotransductive treatment.

According to a further aspect, the first bowel gripping feature and the second bowel gripping feature each include a balloon inflatable between a retracted position and an expanded position.

According to a further aspect, the first bowel gripping feature is disposed on an outer catheter and the second bowel gripping feature is disposed on an inner catheter that is received in the outer catheter.

According to a further aspect, the balloon of the first bowel gripping feature is in communication with a pressurized medium source through a longitudinal aperture that extends through the outer catheter.

According to a further aspect, the balloon of the second bowel gripping feature is in communication with a pressurized medium source through a longitudinal aperture that extends through the inner catheter.

According to a further aspect, a locking seal is configured to engage the inner catheter in a fixed position relative to the outer catheter.

According to a further aspect, the plurality of radially extending fins of the first bowel gripping feature extend angularly in a direction away from the second bowel gripping system and the plurality of radially extending fins of the second bowel gripping feature extend angularly in a direction away from the first bowel gripping system.

According to a further aspect, an atraumatic tip is mounted to an end of the inner catheter adjacent to the second bowel gripping feature.

According to a further aspect, the first bowel gripping feature and the second bowel gripping feature each include a plurality of strips that each support a plurality of the radially extending fins.

According to a further aspect, each of the strips include a base and the plurality of radially extending fins extend from the base at an acute angle.

According to a further aspect, the acute angle is between 20 and 60 degrees.

According to a further aspect, the fins of the first bowel gripping feature and the second bowel gripping feature are made from a flexible medical grade material.

According to a further aspect, the first bowel gripping feature and the second bowel gripping feature each include a gripping material having a base and a plurality of fins extending from the base.

According to a further aspect, the base includes a plurality of slits dividing the gripping material into a plurality of strips that each include a plurality of the radially extending fins.

According to a further aspect, the base is made from a flexible material.

According to a further aspect, the base of the flexible material of the first bowel gripping feature includes a pair of end bands that are connected to the outer catheter and the base of the flexible material of the second bowel gripping feature includes a pair of end bands that are connected to the inner catheter.

According to a further aspect, the end bands include extensions which wrap around the catheter to hold the end band in place, one end of the extension contains a slot and a second end of the extension is tapered to fit through the slot with side lobes wider than the slot to keep the tapered second end engaged through the slot.

According to another aspect, a gripping feature for a distraction device includes a balloon inflatable between a retracted position and an expanded position and configured to be supported on a catheter. A plurality of radially extending fins extend radially outward from the balloon.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a perspective view of a bowel gripping and distraction device according to the principles of the present disclosure;

FIG. 2 is a cross-sectional view of the bowel gripping and distraction device according to the principles of the present disclosure;

FIG. 3 is a top plan view of the bowel gripping and distraction device according to the principles of the present disclosure;

FIG. 4 is a plan view of the bowel gripping portion of the bowel gripping and distraction device according to the principles of the present disclosure in an uninflated state;

FIG. 5 is a plan view of the bowel gripping portion of the bowel gripping and distraction device according to the principles of the present disclosure in an inflated state;

FIG. 6 is a cross-sectional view of the bowel gripping and distraction device according to the principles of the present disclosure;

FIG. 7 is a perspective view of a section of the bowel gripping and distraction device according to the principles of the present disclosure;

FIG. 8 is a cross-sectional view of an end of the inner catheter of the bowel gripping and distraction device according to the principles of the present disclosure;

FIG. 9 is an end view of the inner catheter;

FIG. 10 is a cross-sectional view of the bowel gripping portions of the bowel gripping and distraction device according to the principles of the present disclosure in an uninflated state;

FIG. 11a-11d illustrate the bowel gripping and distraction device in a deactivated state, in an activated state, in an extended state and a deactivated in the extended state, respectively;

FIG. 12 is a side plan view of the inner catheter;

FIG. 13 is a side plan view of the outer catheter;

FIG. 14 is a perspective view of the balloon of the bowel gripping and distraction device in an inflated state;

FIG. 15 is an end plan view of the balloon of the bowel gripping and distraction device in an inflated state;

FIG. 16 is a perspective view of the atraumatic tip of the bowel gripping and distraction device;

FIG. 17 is a cross-sectional view of the atraumatic tip of the bowel gripping and distraction device;

FIG. 18 is a perspective view of the grip material of the bowel gripping and distraction device;

FIG. 19 is a top plan view of the grip material of the bowel gripping and distraction device;

FIG. 20 is a side plan view of the grip material of the bowel gripping and distraction device;

FIG. 21 is a side plan view of the locking seal of the bowel gripping and distraction device; and

FIG. 22 is an exploded perspective view of the locking seal of the bowel gripping and distraction device.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

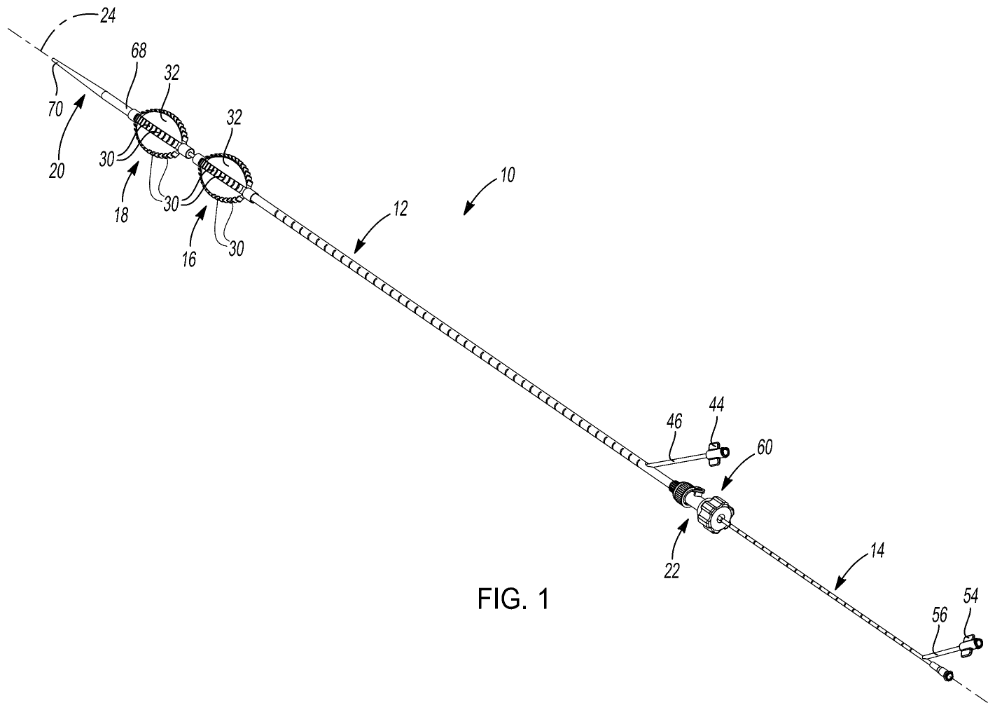

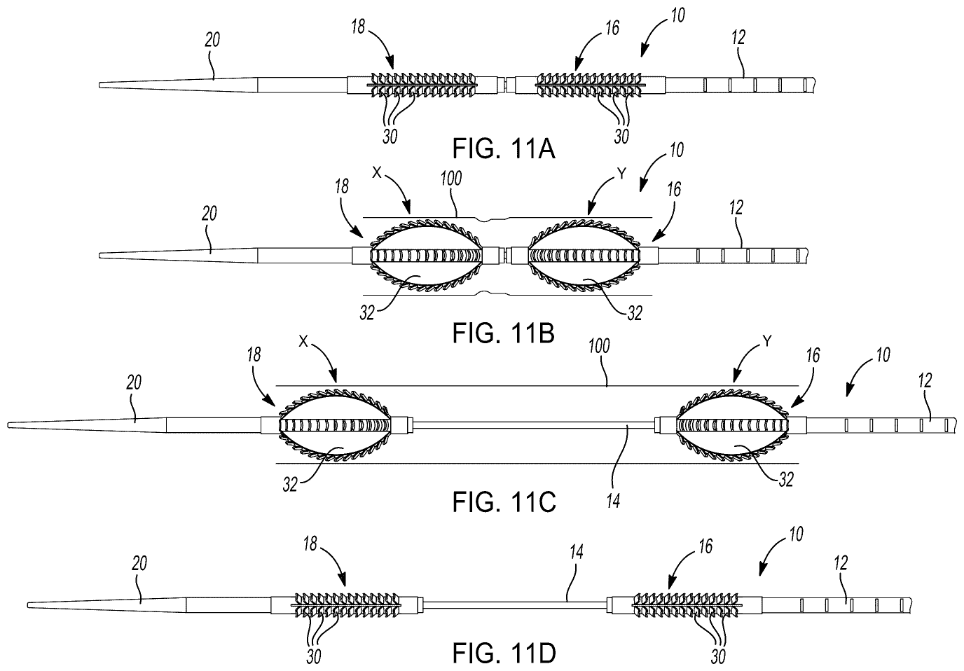

According to the principles of the present teachings, as illustrated in FIG. 1, a bowel gripping and distraction device 10 is disclosed that can be used to correct short bowel syndrome by applying tension to the bowel tissue 100 (FIGS. 11B-11C) which, via the process of mechanotransduction, grows in length in response to the applied tension. Bowel extender device 10 is placed within the small bowel and applies tension over a period of time, typically on the order of several days or weeks, by attaching to the bowel lumen 100 at two different locations (generally denoted at regions X and Y and moving those two points apart, thereby stretching the bowel tissue 100.

With particular reference to FIG. 1, in some embodiments, bowel gripping and distraction device 10 can include an outer catheter 12, an inner catheter 14, a first bowel gripping feature 16 disposed on the outer catheter 12, a second bowel gripping feature 18 disposed on the inner catheter 14, an optional atraumatic tip 20 on an end of the inner catheter 14 and a locking seal 22 for locking a position of the inner catheter 14 relative to the outer catheter 12. A guide wire 24 can assist with placement of the bowel gripping and distraction device 10 and can extend through a center of the inner catheter 14 and the atraumatic tip 20. Generally, the bowel gripping and distraction device 10 is sized and configured to be inserted within the bowel 100 of a mammal, typically human being, for treatment of short bowel syndrome via mechanotransduction.

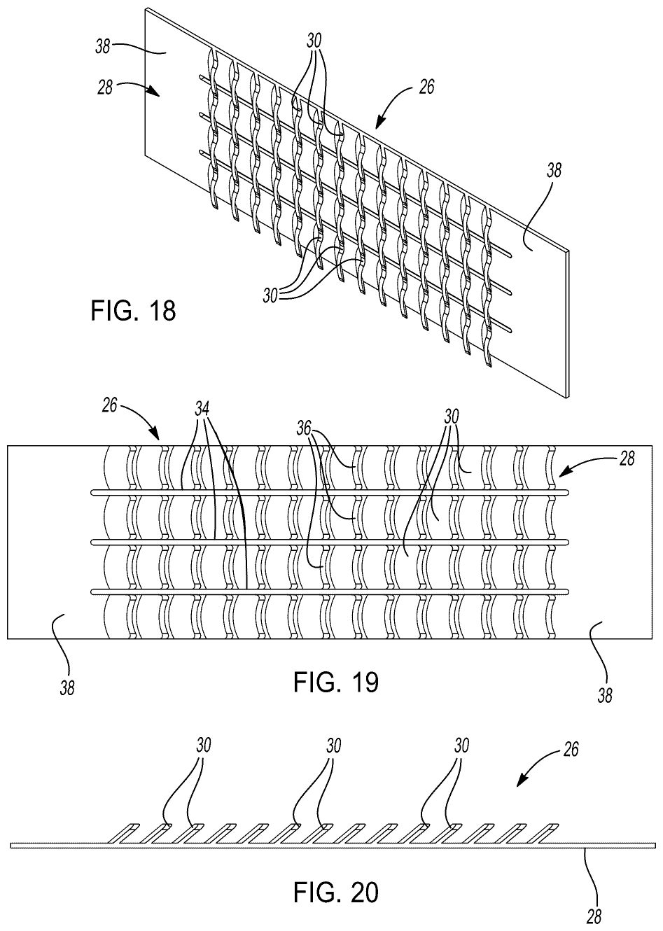

With reference to FIGS. 3 and 18-20, the bowel gripping features 16, 18 each include a gripping material 26 that includes a base 28 and a plurality of angled fins 30. The gripping material 26 of each bowel gripping feature 16, 18 surrounds a respective balloon 32. The gripping material is preferably made of a biocompatible medical grade urethane, TPU, or other flexible material. The wall thickness of the base 28 of the gripping material 26 is preferably such that it is thin enough where balloon inflation within the gripping material 26 causes material elongation of the wall of the gripping material 26. The gripping material 26 can be created in multiple configurations. For example, the gripping material 26 can include a plurality of slits 34 that separates the gripping material into a plurality of strips 36, as shown. Different numbers of strips 36 can be used. These strips 36 could be rectangular or other shapes. The number of strips 36 shown are four although the number of strips 36 can vary. The gripping material 26 could be separate from the balloon 32 or integral to the balloon 32. When the balloon 32 is in a deflated state, the angled fins 30 remain flexible, allowing them to easily deflect and allow the device to translate within the bowel 100, even if the fins 30 are pointing against the direction of travel within the bowel 100. When the balloon 32 is inflated, tension is created on the fin base 28. This tension reduces the fin's ability to deflect, allowing it to more effectively grip the bowel 100. Further, as the balloon 32 inflates, it changes the angle of the fins 30 on the gripping material 26, further improving its ability attach to the bowel 100. Also, as the balloon 32 inflates, it increases the diameter of the gripping material 26, causing it to press more firmly against the bowel 100 (or tissue).

The durometer hardness of the gripping material 26 is preferably between about Shore D 30 to Shore D 70, although other hardness values are envisioned. The base layer 28 for the gripping material shall have sufficient strength such that it does not break during use. However, it is generally preferable to keep the thickness as low as possible to improve device insertion and navigation. The base layer 28 thickness is shown at 0.45 mm. A range of base layer 28 thickness between about 0.2 and 0.6 mm thick is preferable. The fins 30 are preferably angled generally in one direction to improve grip to the bowel 100 for means of applying tension between a given bowel segment. This angle may preferably be between about 20-60 degrees and is shown in FIG. 20 as 40° relative to the base 28. The fins 30 could also be a variety of shapes, including but not limited to rectangular, curved, rounded, triangular, and others. The fins 30 can also be rounded in the direction of the angle. This may help to reduce buckling of the fins 30 when engaged with the tissue/bowel wall 100. The fins 30 may protrude from the surface of the base 28 between approximately 1-3 mm although other distances are contemplated. The fins 30 are preferably between about 0.2 and 0.6 mm thick. The fin material may be of uniform thickness or of varying thickness. This can help further control the deformation profile of the fins 30 and grip material 26. The fins 30 are preferably deformable or of a lower stiffness when in an undeployed position (i.e., when the balloon 32 is not inflated) and are preferably less deformable or of a higher stiffness when in a deployed position (i.e., when the balloon 32 is inflated). In this embodiment that function is created by elongating the base 28 of the unidirectional grip material 26 by inflating a balloon 32. When the base 28 is elongated, the base material surface becomes stiffer, this provides a more stable base 28 for the fins 30 to resist deformation.

The grip material 26 may be manufactured in several ways and configurations. For example, the material may be formed as a series of connected strips 36 connected to end bands 38 which can be wrapped around a catheter (as shown in FIG. 3). The end bands 38 can be glued to the catheter on opposite sides of the balloon 32 of fastened by other means such as shrink fit, clamping, belt buckle extension lobed insert and slot-type and or other connection techniques. The grip material 26 could also be formed as a series of unconnected strips that are circumferentially connected around a balloon/catheter 32. The grip material 26 could also be integrally formed on a balloon 32. The grip material 26 also does not need to be in a straight strip but could alternatively be different spacing around the balloon 32 or different amounts of gripping structures at different points on the balloon 32.

Preferably the balloon 32 is made from a flexible, medical grade material, such as urethane. The balloon can be smooth, or the gripping features can be incorporated directly into the balloon. The balloons 32 ideally inflate to about 35 mm in diameter.

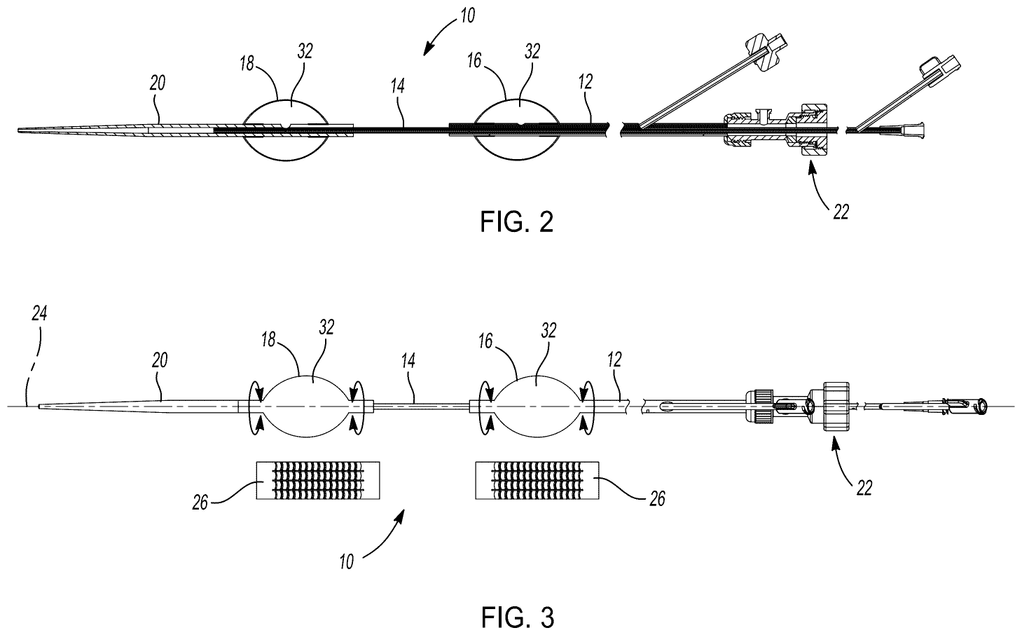

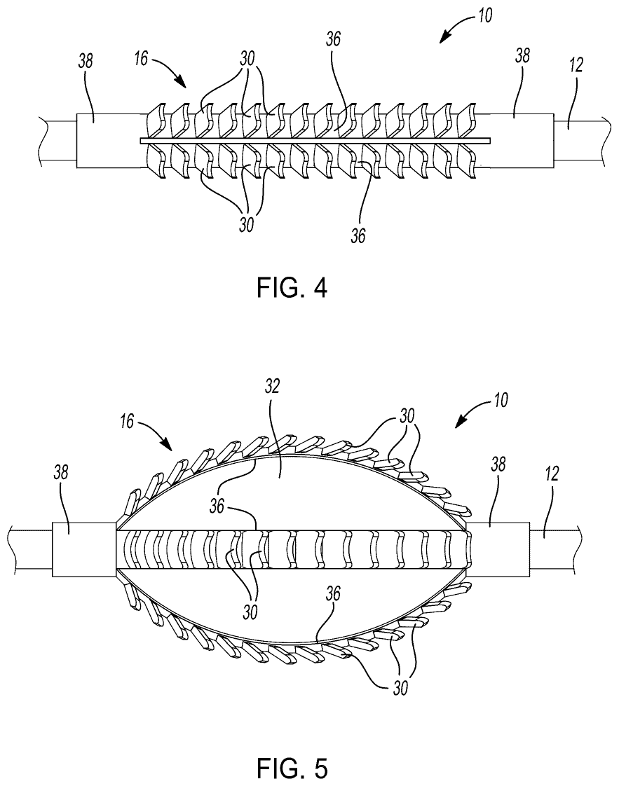

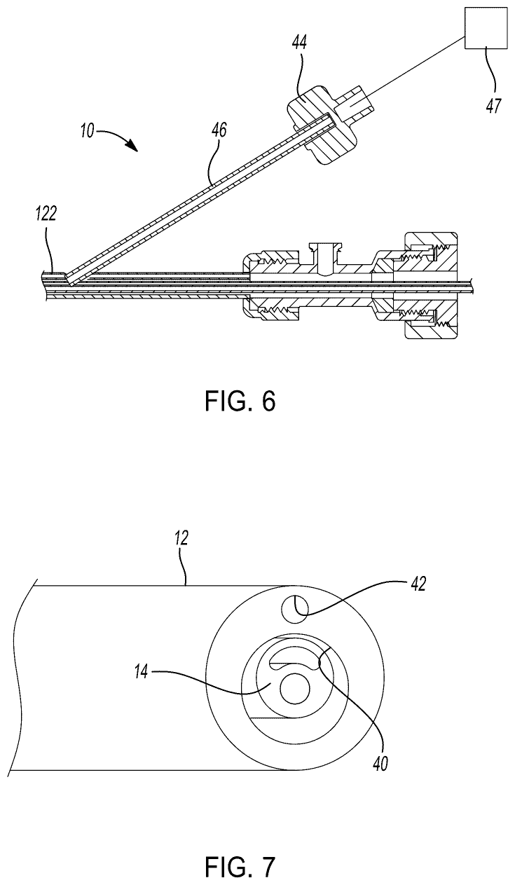

With reference to FIGS. 2, 6 and 7, the outer catheter 12 has a first aperture 40 extending longitudinally therethrough. The inner catheter 14 extends through the first aperture 40. A gap can exist between the outer diameter of the inner catheter 14 and the first aperture 40 of the outer catheter 12. This enables irrigation to be flushed between the catheters 12, 14 which can aid in device use. The first bowel gripping feature 16 is disposed on an end of the outer catheter 12. As shown in FIG. 7, the outer catheter 12 includes a second aperture 42 that is in communication with the balloon 32 within the first bowel gripping feature 16. As shown in FIG. 6, a fitting 44 is connected to an inflation port 46 laterally extending from the outer catheter 12. The inflation port 46 is in communication with the second aperture 42 of the outer catheter 12. A pressurized air source or other pressurized medium source (not shown) is connected to the fitting 44 for supplying a pressurized medium for inflating the balloon 32 with the first bowel gripping feature 16.

The outer catheter 12 is preferably made from a flexible, medical grade material, such as TPU. A hardness range might be between Shore D 10 and Shore D 80. The outer catheter 12 can be optionally radio-opaque to aid with navigation by adding, for example, barium sulfate. The outer catheter 12 can also include lubricious additives to help relative translation between the catheters and tissues.

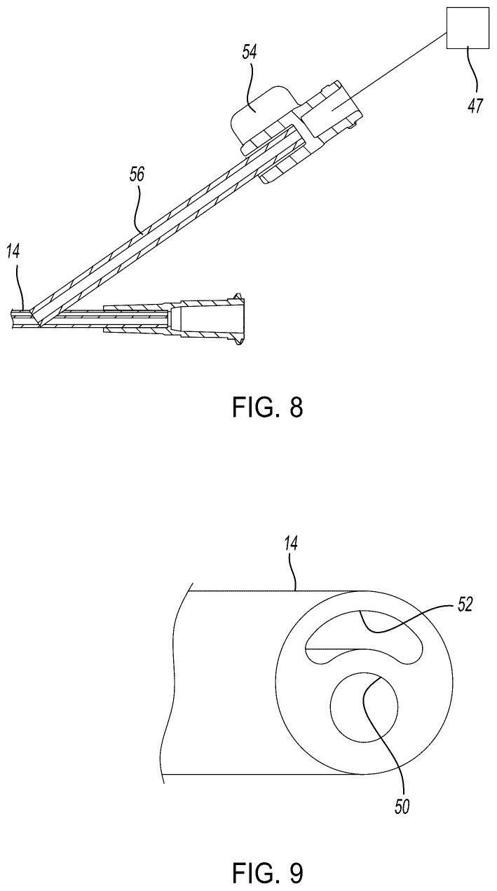

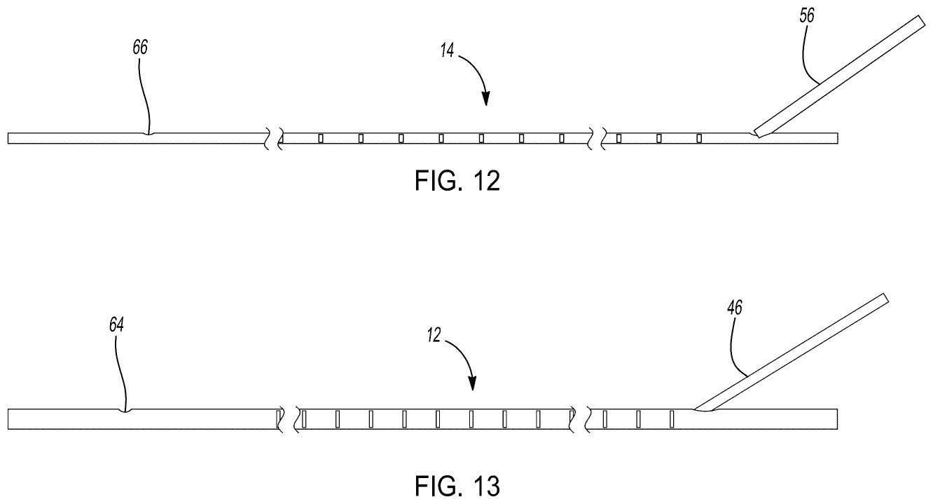

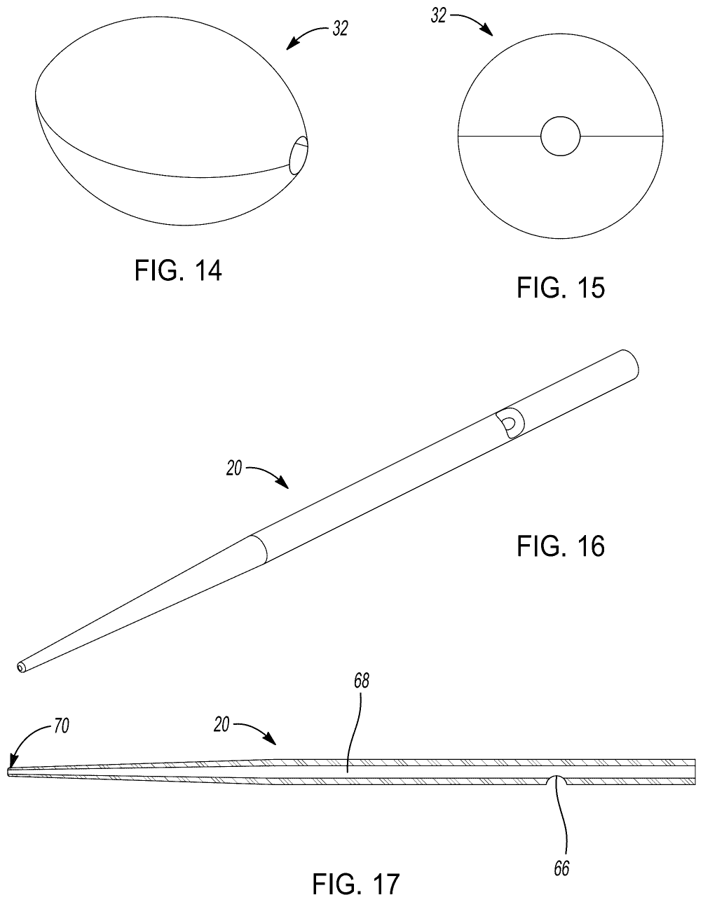

With reference to FIGS. 2, 8, 9 and 12, the inner catheter 14 extends through the first aperture 40 in the outer catheter 12. The inner catheter 14 has a first longitudinal aperture 50 that receives the guide wire 24 therethrough. As shown in FIG. 2, the second bowel gripping feature 18 is mounted to the inner catheter 14. As shown in FIG. 9, the inner catheter 14 includes a second aperture 52 that is in communication with the balloon 32 within the second bowel gripping feature 18. As shown in FIG. 8, a fitting 54 is connected to an inflation port 56 laterally extending from the inner catheter 14. The inflation port 56 is in communication with the second aperture 52 of the inner catheter 14. An pressurized air source or other pressurized medium source (not shown) is connected to the fitting 54 for supplying a pressurized medium for inflating the balloon 32 with the second bowel gripping feature 18.

The inner catheter 14 is preferably made from a flexible, medical grade material, such as TPU. A hardness range might be between Shore D 40 and Shore D 90. The inner catheter 14 can be optionally radio-opaque to aid with navigation by adding, for example, barium sulfate. The inner catheter 14 can also include lubricious additives to help relative translation between the catheters and tissues.

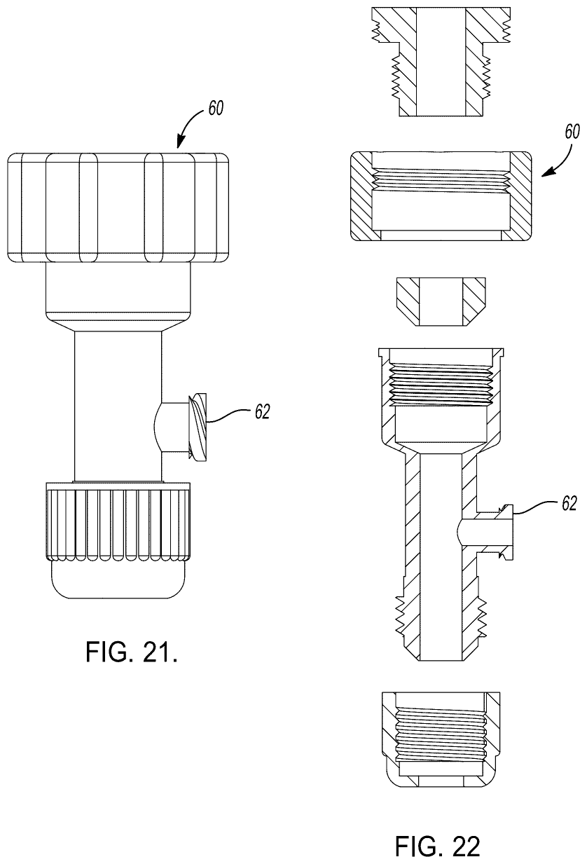

With reference to FIGS. 7, 21 and 22, a locking seal 60 is mounted to a proximal end of the outer catheter 12. The locking seal 60 and the inner and outer catheters 14, 12 provide an elongation system that allows the user to selectively slide and lock the telescoping inner catheter so that the relative distance between bowel gripping features 16, 18 can be changed. The locking seal 60 as shown in FIGS. 21 and 22 is an off the shelf item that is commercially available from Qosina Corp. The locking seal can be, for example, a tuohy borst connector. The locking seal 60 includes a side port 62 that also enables fluid to be flushed between the inner and outer catheters 12, 14.

Optionally, an additional locking seal (not shown) can also be connected to the proximal end of the inner catheter 14 that an enable the relative distance between the inner catheter and the guidewire to be locked in place. This can be beneficial to add stiffness to the inner catheter 14 during bowel elongation by, for example, locking the guidewire 24 in place so the tip resides at least along the outstretched length between the distal and proximal bowel gripping features 16, 18.

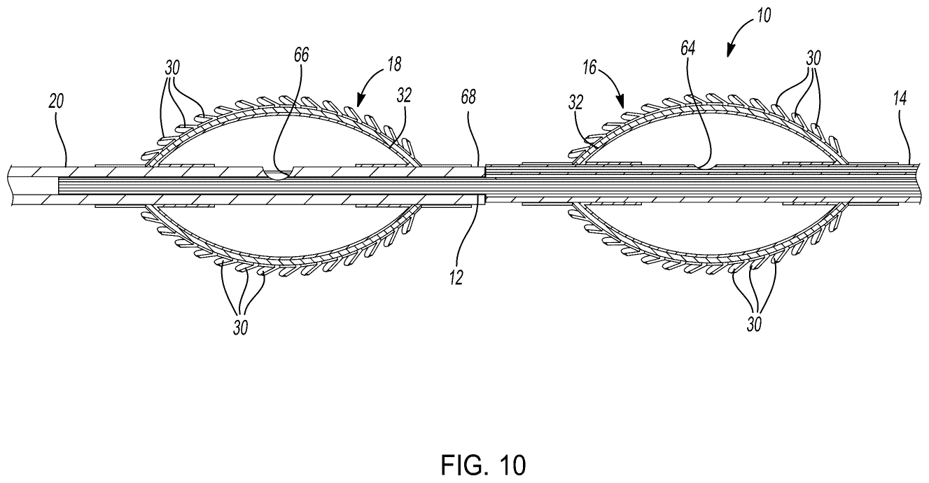

With reference to FIG. 10, the inner and outer catheters 12, 14 can include respective fenestrations 64, 66 to allow the flow of fluid from the inflation ports 46, 56 into the balloons 32.

According to the principles of the present teachings, the atraumatic tip 20 is employed to provide progressive tapering or reduction in the amount of stiffness along at least a portion of the length of the bowel gripping and distraction device 10. More particularly, the stiffness of a proximal end 36 of atraumatic tip 20 that is adjacent an end of the inner catheter 14 is generally greater than a distal end 68 of atraumatic tip 20. In some embodiments, this stiffness reduction along the length of atraumatic tip 20 can be linear, exponential, or define a non-linear predetermined reduction. Atraumatic tip 20 can comprise one or more internal flexure members and an optional covering or sheath disposed about flexure members. The atraumatic tip 20 is preferably made from a flexible, medical grade material, such as TPU. A hardness range of the flexible material can be between Shore A 20 and Shore A 90. The atraumatic tip 20 can have a uniform material hardness/thickness or can be composed of multiple materials and/or thicknesses to create an atraumatic tip 20 with adequate bending properties for the application. The atraumatic tip 20 can optionally be radio-opaque to aid with navigation by adding, for example, barium sulfate.

With reference to FIGS. 11a-11d, the use of the bowel gripping and distraction device 10 will now be described. With reference to FIG. 11a, the bowel gripping features 16, 18 are shown in a deactivated state. With reference to FIG. 11b, the bowel gripping features 16, 18 are shown in an activated (inflated) state. Note that the fin direction on the gripping material 26 is such that the fins 30 of each bowel gripping feature 16, 18 are pointed away from the other bowel gripping feature. Note that the two bowel gripping features 16, 18 do not necessarily have to abut each other in step 2 but could also be separated by some distance prior to activating the bowel gripping features 16, 18. With reference to FIG. 11c, the bowel gripping features 16, 18 are shown inflated and with a relative distance between bowel gripping features 16, 18 is increased, this can act to impart longitudinal tension on the bowel, stimulating growth. With reference to FIG. 11c, the bowel gripping features 16, 18 are deactivated. The device can now be repositioned or removed as desired.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A bowel gripping and distraction device comprising:
a first bowel gripping feature configured to be inflatable to engage a bowel, the first gripping feature including a plurality of radially extending fins;
a second bowel gripping feature configured to be inflatable to engage a bowel, the second gripping feature including a plurality of radially extending fins; and
an elongation system being operable to extend a relative distance between the first bowel gripping feature and the second bowel gripping feature to apply a tensile force to a bowel of a patient, the tensile force being configured to encourage growth of the bowel through mechanotransductive treatment.

2. The bowel gripping and distraction device according to claim 1, wherein the first bowel gripping feature and the second bowel gripping feature each include a balloon inflatable between a retracted position and an expanded position.

3. The bowel gripping and distraction device according to claim 2, wherein the first bowel gripping feature is disposed on an outer catheter and the second bowel gripping feature is disposed on an inner catheter that is received in the outer catheter.

4. The bowel gripping and distraction device according to claim 3, wherein the balloon of the first bowel gripping feature is in communication with a pressurized medium source through a longitudinal aperture that extends through the outer catheter.

5. The bowel gripping and distraction device according to claim 4, wherein the balloon of the second bowel gripping feature is in communication with a pressurized medium source through a longitudinal aperture that extends through the inner catheter.

6. The bowel gripping and distraction device according to claim 3, further comprising a locking seal configured to engage the inner catheter in a fixed position relative to the outer catheter.

7. The bowel gripping and distraction device according to claim 1, wherein the plurality of radially extending fins of the first bowel gripping feature extend angularly in a direction away from the second bowel gripping system and the plurality of radially extending fins of the second bowel gripping feature extend angularly in a direction away from the first bowel gripping system.

8. The bowel gripping and distraction device according to claim 3, further comprising an atraumatic tip mounted to an end of the inner catheter adjacent to the second bowel gripping feature.

9. The bowel gripping and distraction device according to claim 1, wherein the first bowel gripping feature and the second bowel gripping feature each include a plurality of strips that each support a plurality of the radially extending fins.

10. The bowel gripping and distraction device according to claim 9, wherein each of the strips include a base and the plurality of radially extending fins extend from the base at an acute angle.

11. The bowel gripping and distraction device according to claim 1, wherein the fins of the first bowel gripping feature and the second bowel gripping feature are made from a flexible medical grade material.

12. The bowel gripping and distraction device according to claim 1, wherein the first bowel gripping feature and the second bowel gripping feature each include a gripping material having a base and a plurality of fins extending from the base.

13. The bowel gripping and distraction device according to claim 12, wherein the base includes a plurality of slits dividing the gripping material into a plurality of strips that each include a plurality of the radially extending fins.

14. The bowel gripping and distraction device according to claim 12, wherein the base is made from a flexible material.

15. The bowel gripping and distraction device according to claim 12, wherein the base of the flexible material of the first bowel gripping feature includes a pair of end bands that are connected to the outer catheter and the base of the flexible material of the second bowel gripping feature includes a pair of end bands that are connected to the inner catheter.

16. The bowel gripping and distraction device according to claim 15, wherein the end bands include extensions which wrap around the catheter to hold the end band in place, one end of the extension contains a slot and a second end of the extension is tapered to fit through the slot with side lobes wider than the slot to keep the tapered second end engaged through the slot.

17. A gripping feature for a distraction device comprising:
a balloon inflatable between a retracted position and an expanded position and configured to be supported on a catheter; and
a plurality of radially extending fins extending radially outward from the balloon.

18. The gripping feature according to claim 17, wherein the plurality of radially extending fins are supported on a plurality of strips, each of the strips include a base and the plurality of radially extending fins extend from the base at an acute angle.

19. The gripping feature according to claim 18, wherein the acute angle is between 20 and 60 degrees.

20. The gripping feature according to claim 17, wherein the plurality of fins are supported on a flexible base.
